(19) United States
(12) Patent Application Publication
(10) Pub. No.: US 2013/0122539 A1
(43) Pub. Date: May 16, 2013

(54) MICROSIEVE FOR CELLS AND PARTICLES FILTRATION

(71) Applicants: Mo-Huang Li, Singapore (SG); Min Hu, Singapore (SG); Wal Chye Cheong, Singapore (SG); Tau Liang Gan, Singapore (SG)

(72) Inventors: Mo-Huang Li, Singapore (SG); Min Hu, Singapore (SG); Wal Chye Cheong, Singapore (SG); Tau Liang Gan, Singapore (SG)

(21) Appl. No.: 13/696,064

(22) PCT Filed: May 3, 2011
(86) PCT No.: PCT/SG2011/000173

§ 371 Date: Jan. 22, 2013

(30) Foreign Application Priority Data
May 4, 2010 (SG) 201003161-5

(51) Int. Cl. C12M 1/26

(52) U.S. Cl. CPC C12M 33/14
USPC 435/34; 435/325; 422/535; 430/320

(57) ABSTRACT

It is disclosed a microsieve comprising two layers, wherein the first layer is a membrane layer having a plurality of micropores contained therein and a thickness of about 10 μm to about 100 μm, and the second layer is a membrane support layer having a plurality of openings contained therein and a thickness of about 100 μm to about 500 μm, wherein the openings are larger in diameter than the micropores, and wherein at least one of the membrane layer or membrane support layer is formed of a SU-8 photoresist material.

### CROSS-REFERENCE TO RELATED APPLICATION

This application claims the benefit of priority of Singapore Patent Application No. 201003161-5, filed 4 May 2010, the contents of which being hereby incorporated by reference in its entirety for all purposes.

### TECHNICAL FIELD

The invention relates to a microsieve, and in particular, to a microsieve having two asymmetric layers for cells and particles filtration.

### BACKGROUND

The isolation of specific components from liquid samples has become increasingly important not only for research purposes but also for diagnostics in clinical laboratories. In particular for clinical applications, systems are needed which can determine the presence or absence of certain components in samples obtained from a patient in a fast and reliable manner which allows a clinician to make a diagnosis and determine the further treatment of a patient. Such systems are also need in research laboratories.

For example, circulating tumor cells (CTCs) are very rare in peripheral blood of cancer patients, as few as one cell per 10⁹ haematologic cells in the blood of cancer patients with metastatic cancer. The number of these cells has been shown to correlate with the disease development, and represents a potential alternative to invasive biopsies for cancer metastasis analysis. Several technologies have been developed to isolate the CTCs from cancer patients' blood samples, such as density gradient centrifugation, immunomagnetic method (MACS), ferrofluid method (CellSearch™), and cell filtration by microsieves.

Among the various cell isolation techniques, cell filtration appears to be most promising and attractive since less sophisticated equipments are required and faster response time or isolation time is attainable. Current cell filtration techniques include the use of microsieves made of silicon, parylene or polycarbonate. However, such microsieve materials suffer disadvantages such as incompatibility with cell culture reagents, high material costs, and low pore density, to name a few.

Therefore, there is a need to develop a microsieve to overcome or at least alleviate the above problems for rapid cell and particle filtration.

### SUMMARY

Various embodiments provide for a low-cost and disposable microsieve for efficient cells filtration in a fluid. The microsieve is designed with two asymmetric layers. The microsieve may be fabricated by a conventional double-layer lithography process, which enables control of precision and uniformity of the micropores to be formed while at the same time affords mass fabrication capability. In addition, the microsieve minimizes flow resistance, resulting in a high trans-membrane flux, therefore tremendously reduces cell separation time.

Various embodiments provide for a microsieve comprising two layers, wherein:
the first layer is a membrane layer having a plurality of micropores contained therein and a thickness of about 10 μm to about 100 μm; and
the second layer is a membrane support layer having a plurality of openings contained therein and a thickness of about 100 μm to about 500 μm, wherein the openings are larger in diameter than the micropores, and wherein at least one of the membrane layer or membrane support layer is formed of a SU-8 photoresist material.

Various embodiments also provide for a method of preparing a microsieve, comprising:
providing a substrate;
coating the substrate with a first layer having a thickness of about 10 μm to about 100 μm;
patterning the first layer to form a plurality of micropores therein;
coating the patterned first layer with a second layer having a thickness of about 100 μm to about 500 μm; and
patterning the second layer to form a plurality of openings wherein the openings are larger in diameter than the micropores, and wherein at least one of the first layer or second layer is formed of a SU-8 photoresist material.

Various embodiments further provide for a device for separating cells of a defined size from a fluid sample, where the device comprises:
an inlet module having an inlet for the fluid sample entry;
an outlet module having an outlet for the fluid sample exit;
a microsieve of the above various embodiments having micropores for retaining cells of a defined size arranged between the inlet module and the outlet module, wherein the inlet module, the outlet module, and the microsieve are fluidly connected to each other to allow the fluid sample to pass through from the inlet module to the outlet module.

Various embodiments further provide for a method of separating cells of a defined size from a fluid sample comprising filtering the fluid sample suspected to comprise a cell to be separated through an inlet of the device of the above various embodiments.

Various embodiments provide the use of the device of the above various embodiments for filtering blood.

### BRIEF DESCRIPTION OF THE DRAWINGS

In the drawings, like reference characters generally refer to the same parts throughout the different views. The drawings are not necessarily drawn to scale, emphasis instead generally being placed upon illustrating the principles of various embodiments. In the following description, various embodiments of the invention are described with reference to the following drawings.

FIG. 1 shows (a) operation principle of circulating tumor cells (CTCs) enrichment; (b) principle of cell purification from dissociated tissue.

FIG. 2 shows the fabrication process of SU-8 microsieve. (a) Spin coating of lift-off resist 10B. (b) Spin coating and patterning of first SU-8 layer for filtration membrane layer. (c) Spin coating and patterning of second SU-8 layer for membrane support layer. (d) Develop. (e) Lift-off the SU-8 microsieve from the silicon substrate. The insert shows the design of SU-8 microsieve.

FIG. 3 shows (a) SEM of microsieve structure. (b) Cross section of 10-μm diameter through channels. (c, d) Optical images of highly uniform 10-μm and 25-μm diameter SU-8 microsieves.

FIG. 4 shows fluorescence images of filtrated HepG2/GFP tumor cells. HepG2/GFP cells are spiked into 1-ml undiluted rabbit whole blood and filtrated with 10-μm diameter SU-8 microsieve membrane. (a) Cell nucleus stained with DAPI (blue). (b) Morphology of HepG2/GFP (green).

FIG. 5 shows flow rate versus the filtration time of 25-μm diameter SU-8 microsieve with variant filtration conditions. (a) 4.5 ml PBS buffer with 1.5 kPa pressure. (b,c) 4.5 ml rabbit whole blood with 5 kPa pressure on plasma treated (b) or un-treated (c) SU-8 microsieves. The insert shows the experimental setup.

FIG. 6 shows the schematic of SU-8 microsieve device for drug response study. (a) Circulating tumor cells (CTCs) are filtrated using SU-8 microsieve. (b) Captured CTCs are cultured on a cell culture medium. (c) Cells in separate wells are treated with drugs. The cells' responses are monitored with a microscope. In Design (A), the structure of microsieve supporting rings is utilized as physical walls of microwells.

FIG. 7 shows the schematic of the SU-8 microsieve integrated with a plastic holder. (a,b) The plastic holder consists of one outlet. (c,d) The plastic holder consists of an outlet with a Duckbill check valve. (e,f) The plastic holder consists of an outlet with small holes. The supporting rings of microsieve are either upwards or downwards. (g) A device incorporating the microsieve sandwiched between two modules.

### DESCRIPTION

The following detailed description refers to the accompanying drawings that show, by way of illustration, specific details and embodiments in which the invention may be practised. These embodiments are described in sufficient detail to enable those skilled in the art to practise the invention. Other embodiments may be utilized and structural, logical, and electrical changes may be made without departing from the scope of the invention. The various embodiments are not necessarily mutually exclusive, as some embodiments can be combined with one or more other embodiments to form new embodiments.

Various embodiments provide for a low cost, disposable microsieve with controlled micropore diameters for applications such as circulating tumor cells (CTCs) isolation from whole blood and epithelial cells purification from dissociated tissue solution. Fluid regulation and cell counting may be achieved by a simple vacuum pumping and laboratory fluorescence microscope on a ready-to-use filter unit which is simpler compared to the existing CTCs isolation methods. The microsieve offers new solutions for rapid rare cells filtration from clinical sample volume of as low as 5 ml.

In various embodiments, the microsieve may include two layers.

The first layer may be a membrane layer having a plurality of micropores contained therein. The membrane layer serves to filter cells (or other particles) contained in a fluid sample so that the cells may be collected and collated for further analysis.

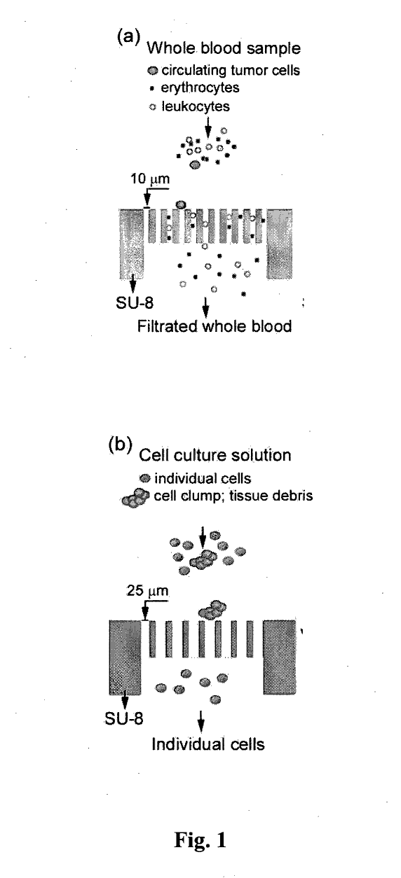

Depending on the specific application and the type of cells to be analyzed, the diameter of the micropores may be tailored and controlled accordingly. In various embodiments, each of the plurality of micropores has a pore diameter of about 5 μm to about 50 μm. In one embodiment as illustrated in FIG. 1(a), for CTC filtration the pore diameter of each micropore may be fabricated to be about 10 μm and having a 15 μm pitch (i.e. distance between the centre of two neighbouring micropores). In another embodiment as illustrated in FIG. 1(b), for dissociated epithelial cells purification the pore diameter of each micropore may be fabricated to be about 25 μm and having a 30 μm pitch. It is to be understood and appreciated that the size of the micropores in the membrane layer is to be designed to be smaller than the size of the cells to be retained by the micropores while allowing the fluid sample including other particles or entities to pass through.

In various embodiments, the micropores may be formed in the shapes of circle, square, rectangle, triangular, polygonal, or any other regular or irregular configuration. In one embodiment, the micropores are circular. The term “diameter” as used in the current description is understood to mean the maximum distance between any pair of points in the shape or configuration. For example, diameter of a square in this case refers to the diagonal length.

In various embodiments, the membrane layer may have a thickness of about 10 μm to about 100 μm. One the one hand, if the thickness of the membrane layer is too thin, the perforated membrane layer may be physically too weak or fragile and may rupture easily during CTCs filtration of whole blood, for example. A whole blood sample containing white blood cells, red blood cells, and platelets is more viscious than water and hence more force is needed to pump the whole blood sample through the membrane layer. Thus, the thickness of the membrane layer is advantageously selected to be able to withstand the pressure exerted by the fluid sample as it passes through the micropores of the membrane layer.

Another disadvantage of having too thin a membrane layer thickness is that achieving a smooth and flat surface of the membrane layer exposed to the influx of the fluid sample becomes increasingly difficult. In various embodiments, to reduce the processing time the isolated cells retained in the micropores of the microsieve may be directly stained with different fluorescence dyes and counted using fluorescence microscope on which the microsieve is placed. In such applications, the need for a smooth and flat membrane layer surface becomes more apparent. In addition, the microsieve may be surface-coated with a metal layer. SU-8 photoresist materials possess fluorescence and thus may interfere with the fluoroscence counting of cells. By coating a metal layer on the microsieve surface, the fluorecence noise from the SU-8 photoresist materials may be minimized.

On the other hand, if the thickness of the membrane layer is too thick, the fluid resistance may pose a severe problem to the rate of separation of cells in the fluid sample due to the longer distance in the micropores the fluid sample has to pass through and therefore longer time will be needed for the filtration. Cells retained by the micropores create a barrier to the fluid sample passing through the micropores. As more cells get retained, the availability of micropores for fluid sample passage becomes lesser and fluid resistance becomes increasingly severe. To further address the fluid resistance issue, the microsieve may be surface-treated to decrease fluid resistance. In one embodiment, the microsieve surface may be treated with a low pressure plasma technology via electromagnetic discharge of gas at low temperature.

In addition, a thick membrane layer may translate to unnecessary material cost and wastage of resources, and is therefore not commercially and environmentally desirable.

In various embodiments, the membrane layer may be selected to have a thickness of about 50 μm to about 100 μm.

The second layer of the microsieve may be a membrane support layer having a plurality of openings contained therein. The openings of the membrane support layer are made larger in diameter than the micropores of the membrane layer. The membrane support layer serves as a physical support to the membrane layer while at the same time minimizes fluid resistance as the fluid sample passes through the micropores of the membrane layer and the openings of the membrane support layer.

In various embodiments, the openings may be formed in the shapes of circle, square, rectangle, triangular, polygonal, hexagonal, or any other regular or irregular configuration. In one embodiment, the openings are hexagonal. The term “diameter” as used in the current description is understood to mean the maximum distance between any pair of points in the shape or configuration. For example, diameter of a square in this case refers to the diagonal length.

As already noted above, the thickness of the membrane layer must be carefully selected and cannot be too thin or too thick. The thickness of the membrane support layer is correspondingly selected to provide mechanical strength to the membrane layer such that the microsieve is able to withstand the pressure exerted by the fluid sample as it passes through the micropores of the membrane layer and the openings of the membrane support layer.

In various embodiments, the membrane support layer may have a thickness of about 100 μm to about 500 μm. For example, the membrane support layer may have a thickness of about 200 μm to about 300 μm. If the thickness of the membrane support layer is too thick, it may translate to unnecessary material cost and wastage of resources, and is therefore not commercially and environmentally desirable.

The openings in the membrane support layer are designed to be larger than the size of the micropores in the membrane layer so that fluid resistance may be reduced. Fluid sample passing through the smaller micropores experiences higher pressure than passing through the larger openings. Hence, the openings in the membrane support layer alleviates the high fluid resistance problem by providing a larger volume of space for the fluid sample to pass through.

In various embodiments, the openings in the membrane support layer are at least 10 times larger in diameter than the micropores in the membrane layer.

In various embodiments, at least one of the membrane layer or membrane support layer or both is formed of a SU-8 photoresist material. The advantages of employing SU-8 photoresist materials for the membrane layer and/or membrane support layer include its high mechanical strength, biocompatibility with cell culture reagent, compatibility for fabrication by conventional photolithographic techniques, low material costs, hydrophobicity, high chemical and thermal stability, to name a few.

In one embodiment, both the membrane layer and the membrane support layer are formed of SU-8 photoresist material.

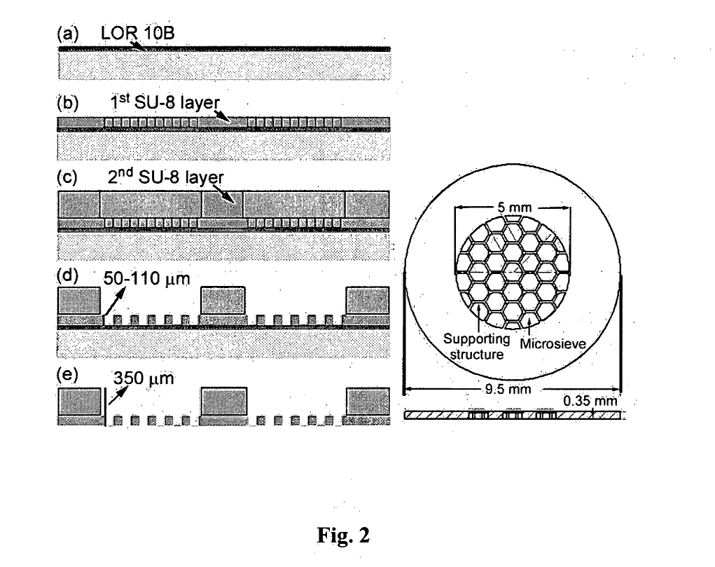

FIG. 2(a)-(e) illustrates in various embodiments a method of preparing a microsieve of the present invention.

The method may include providing a substrate. In one embodiment, the substrate may be silicon. The substrate may be cleaned in a piranha solution to remove organic contaminants on the substrate surface.

The substrate is then coated with a first layer having a thickness of about 10 μm to about 100 μm. In one embodiment, the first layer may be formed of a SU-8 photoresist material. In one embodiment, the first layer may be spin-coated onto the substrate.

The first layer is subsequently patterned to form a plurality of micropores therein. Patterning of the first layer may include applying a photoresist mask that defines a pattern of dots corresponding to the micropores to be formed, exposing the first layer to light, and removing the photoresist mask. Conventional photolithography apparatus and methodology may be employed. For example, the photoresist mask may be a chrome-coated quartz mask having a pattern of circular features and UV-lithography may be carried out at about 365 nm wavelength to transfer the mask features to the first layer to form a perforated membrane layer. While circular micropores are illustrated in FIG. 2, it is understood and appreciated that the shape of the micropores is not limited to such configurations, as mentioned in previous paragraphs above.

A second layer may be coated onto the patterned first layer. The second layer may have a thickness of about 100 μm to about 500 μm. In one embodiment, the second layer may be formed of a SU-8 photoresist material. In one embodiment, the second layer may be spin-coated onto the first layer.

The second layer is subsequently patterned to form a plurality of openings therein. The openings are larger in diameter than the micropores in the membrane layer. Patterning of the second layer may include applying a photoresist mask that defines a pattern of shapes corresponding to the openings to be formed, exposing the second layer to light, and removing the photoresist mask. Conventional photolithography apparatus and methodology may be employed. For example, the photoresist mask may be a plastic mask having a pattern of hexagonal features and UV-lithography may be carried out at about 365 nm wavelength to transfer the mask features to the second layer to form a perforated membrane support layer. While hexagonal or honeycomb rings or openings are illustrated in FIG. 2, it is understood and appreciated that the shape of the openings is not limited to such configurations, as mentioned in previous paragraphs above.

In various embodiments, the substrate may be first coated with a lift-off resist layer prior to coating the first layer. In one embodiment, the lift-off resist layer may be spin-coated onto the substrate. The first layer is subsequently coated onto the lift-off resist layer.

After patterning the first layer and the second layer, the microsieve may be developed in a developing solution. Any suitable SU-8 developer solution may be used.

After developing, the lift-off resist layer and/or the substrate may be removed to obtain the microsieve having the two asymmetric layers.

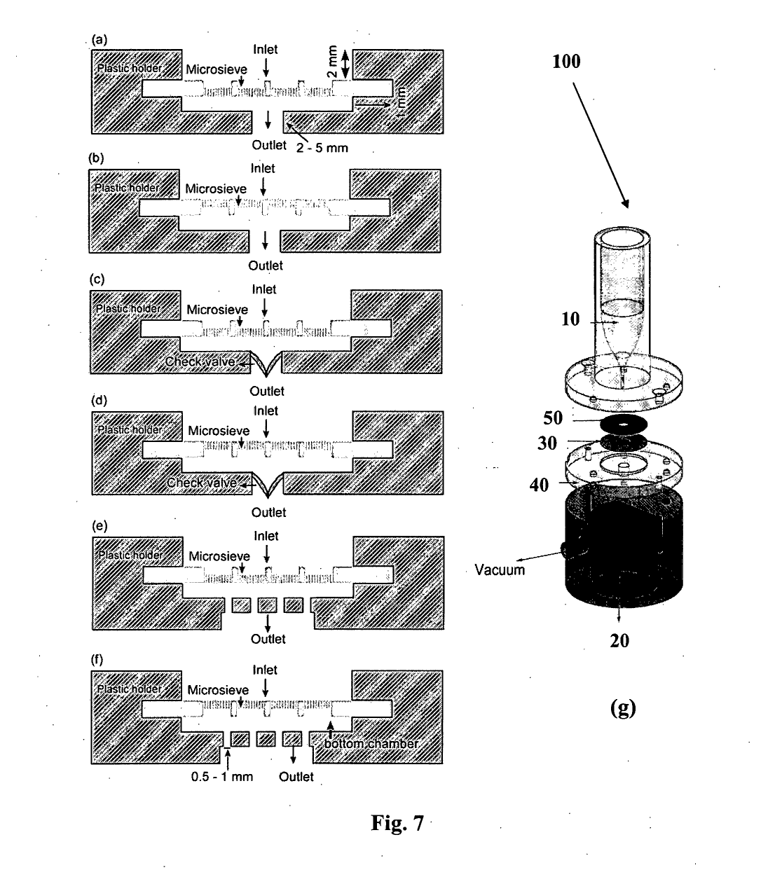

Various embodiments provide for a device 100 for separating cells of a defined size from a fluid sample, which device is illustrated in FIG. 7(g). The device 100 may include an inlet module 10 having an inlet for the fluid sample entry and an outlet module 20 having an outlet for the fluid sample exit. The device 100 further comprises a microsieve 30 of the various embodiments described previously having micropores for retaining cells of a defined size arranged between the inlet module 10 and the outlet module 20, wherein the inlet module 10, the outlet module 20, and the microsieve 30 are fluidly connected to each other to allow the fluid sample to pass through from the inlet module 10 to the outlet module 20.

The device 100 may further include a microscope viewing plate 40 such as a fluorescence microscope onto which the microsieve 30 may be placed. In one embodiment, the inlet module 10 and the outlet module 30 are removably connected and thereby separable so that the microsieve which is supported by the microscope viewing plate 40 may be stained with fluorescence dye for cell counting.

The device 100 may further include a gasket placed between the inlet module 10 and the microsieve 30. Vacuum may also be applied to the outlet module 20 to aid in the drawing of fluid sample through the microsieve.

The device may be used for filtering whole blood. In one embodiment, the device may be used for separating circulating tumor cells from whole blood.

Various embodiments provide for a method of separating cells of a defined size from a fluid sample. The method may include filtering the fluid sample suspected to comprise a cell to be separated through an inlet module of the device described above.

In various embodiments, the fluid sample is selected from the group consisting of whole blood, urine, culture medium, and lysed tissue solution.

In various embodiments, the cells to be detected are selected from the group consisting of circulating tumor cells, epithelial cells, cancer cells or cancer stem cells from lysed cancer tissue, cells comprised in a urine sample, and enrichment of cells from cell culture medium.

A low cost microsieve with unique two asymmetric layer which balances the fluid resistance and the mechanical strength of the device has been demonstrated. In particular, the fluid resistance has been minimized, resulting in a high trans-membrane flux, which tremendously reduces cell separation time. Successful CTCs isolation has been demonstrated from undiluted whole blood sample with spiked cancer cells. Using SU-8 as structure material for both layers allows mass fabrication of the microsieves with precise pore size at feasible cost of dimes per device since SU-8 material is lower in cost as compared to glass capillary array, silicon-based microsieves and microfabricated parylene membrane. The pore size of SU-8 microsieve can be optimized for various cells and particles separation based on the dimensions of target cells. This approach may be suitable for tumor cells isolation from patient whole blood, and cancer diagnosis, and may be extended to the isolation and detection of other cells from whole blood for various disease diagnoses such as CD4+ T cells for HIV testing, fetal cells isolation from maternal blood, and non-invasive prenatal diagnosis, as well as removal of cell aggregates and large particles in organ printing and cell seeding.

In order that the invention may be readily understood and put into practical effect, particular embodiments will now be described by way of the following non-limiting examples.

### EXAMPLES

### Example 1

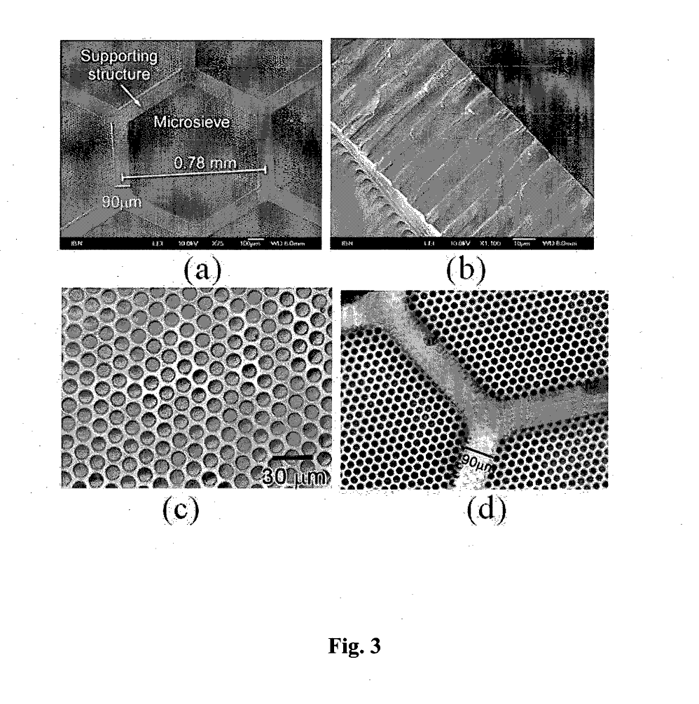

More than 60 microsieves (each having a 1-cm diameter) may be fabricated per run by using a double-layer SU-8 microfabrication process on a 4″ diameter silicon substrate described in the method above. FIG. 3 shows the highly uniform micropore structure and smooth through-hole surface of the densely packed micropore array. Unlike conventional in-plane microsieves which usually have limited pore density (approximately 200 pores/mm²), the present vertical microsieve contains approximately 5,000 pores/mm² with pore opening of more than 40% of the microsieve area, allowing faster CTCs filtration.

### Example 2

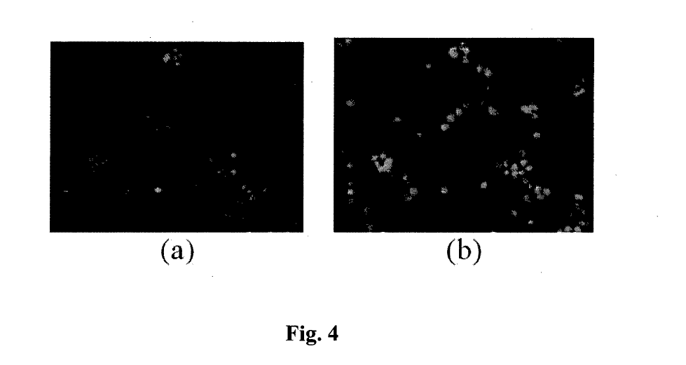

FIG. 4 shows the filtration of CTCs using 10-μm diameter SU-8 microsieves, demonstrated by spiking HepG2/GFP cancer cells (liver carcinoma) into 1-ml undiluted rabbit whole blood to mimic the clinical samples. Captured HepG2/GFP cells are stained with DAPI (nucleus) and inspected with a fluorescence microscope. These images clearly demonstrate that the captured CTCs are immobilized on the smooth and flat surface of the microsieve, which simplifies the imaging process for tumor cells classification and enumeration.

### Example 3

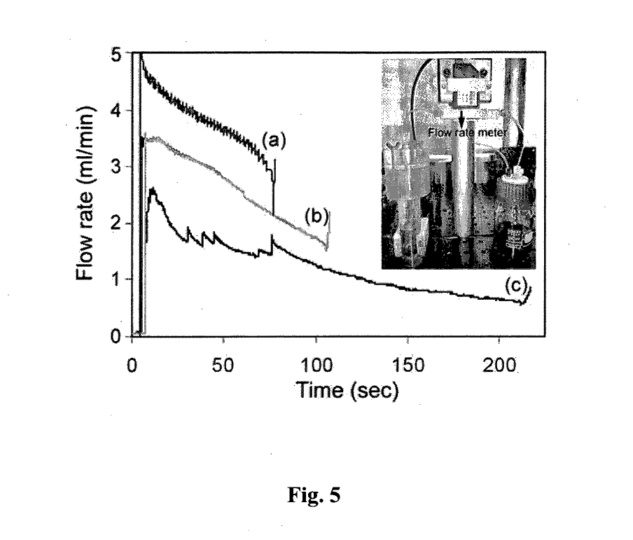

SU-8 microsieve was treated using EuroPlasma CD3000 to alter its surface properties. This system utilized low pressure plasma technology via electromagnetic discharge of gas at low temperature. The plasma interacts with the SU-8 surface and changes its surface properties. This plasma treatment was carried out at a base pressure of 10 mTorr with an electromagnetic power of 100 W, and with oxygen (O₂) and methane (CH₄) induced plasma for 5 and 10 min. Some experiments were conducted with an O₂ plasma pre-treatment for 10 min. Substantial reduction (approximately 60°) in surface contract angle was observed after surface treatment (see Table 1). This surface treatment reduces the fluid resistance of SU-8 microsieve, resulting in a rapid whole blood filtration as shown in FIG. 5. Other surface treatments such as coating the fabricated microsieve with a bio-compatible parylene thin film (less than 5 μm), anti-fouling poly(ethylene glycol)-silanes, extracellular matrix materials, and Matrigel can also be applied.

The SU-8 microsieve has extremely low fluid resistance. The undiluted whole blood (4.5 ml) is effectively filtrated within 4 min with the microsieve subjected to a 5 kPa vacuum pressure (FIG. 5(c)). The filtration time is further reduced to less than 2 min (FIG. 5(b)) when the device is treated with an oxygen/methane plasma. Table 2 compares the calculated pressure drop (ΔP_{n-ch}) and maximum loading pressure (Pₘₐₓ) of known silicon, parylene, polycarbonate, and present SU-8 CTCs microsieves. The SU-8 microsieve with 2-layer structure provides 52× and 89× higher mechanical strength than that of polycarbonate and parylene, respectively.

TABLE 1
Surface contact angle of SU-8 microsieve
with various plasma treatment conditions

| | | With 10 min | With 10 min | Without | Without |
|---|---|---|---|---|---|
| | | O₂ pre- | O₂ pre- | O₂ pre- | O₂ pre- |
| | | treatment. | treatment. | treatment. | treatment. |
| | Without | 5 min | 10 min | 5 min | 10 min |
| | treatment | treatment | treatment | treatment. | treatment |
| SU-8 1 | 82.1° | 23.0° | 22.4° | 23.7° | 19.5° |
| SU-8 2 | 88.1° | 21.9° | 22.1° | 24.3° | 25.8° |
| SU-8 3 | 81.1° | 22.1° | 22.5° | 23.3° | 17.1° |

TABLE 2
Comparison of membrane material for CTCs filtration

| | E | σ_{yield} | h | l | | Pₘₐₓ | ΔP_{n-ch} |
|---|---|---|---|---|---|---|---|
| Material | (GPa) | (MPa) | (μm) | (mm) | p | (10⁴ Pa) | (Pa) |
| Parylene C | 4.5 | 59 | 6 | 5 | 0.3 | 0.33 | 50 |
| SU-8 | 5 | 100 | 60 | 1 | 0.4 | 29.4 | 172 |
| Polycarbonate | 2.6 | 70 | 10 | 5 | 0.15 | 0.56 | 125 |
| Silicon | 190 | 7000 | 100 | 5 | 0.4 | 936 | 277 |

Pressure drop (ΔP_{n-ch}) of n identical parallel channels and the maximum loading pressure of an perforated membrane (Pₘₐₓ) [10] are given by:
$Δ {P}_{n - ch} = \frac{μ Q}{{n ( d / 2 )}^{3}} [ \frac{16}{π} ( \frac{h}{d} ) + 3 ] and {P}_{max} \approx 0.58 \frac{h {σ}_{yield}^{3 / 2}}{{lE}^{1 / 2}} ( 1 - p )$ h: channel length;
Q: flow rate (1 ml/min) ;
p: viscosity of the carrier (3.8 cps);
d: channel diameter (10 μm);
E: Young's modulus;
σ_{yield}: yield stress;
l: the distance between supporting bars;
p: porosity of membrane.

### Example 4

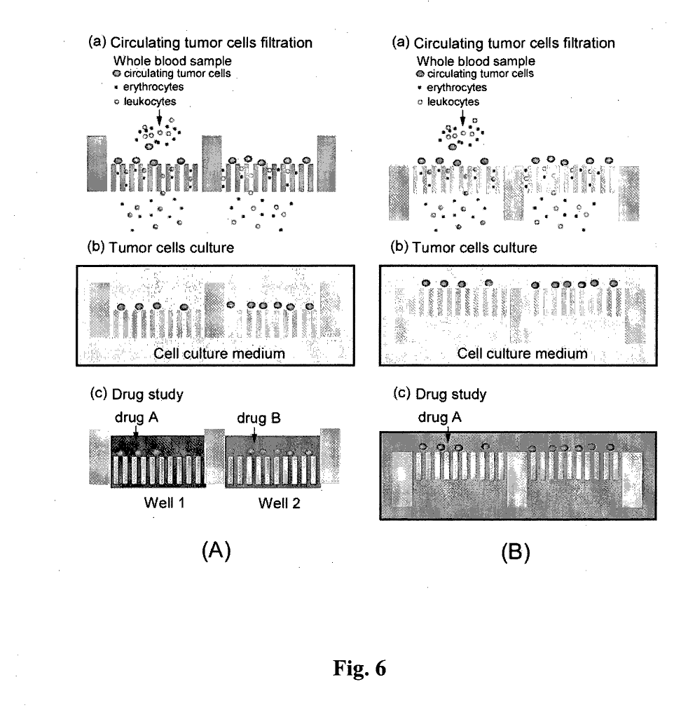

Beside the circulating tumor cells filtration, the SU-8 microsieve can also be used for on-microsieve cell culture, and cancer drug study as shown in FIG. 6. The captured CTCs on the microsieve surface are transported to a cell culture medium, and incubated in an incubator overnight. The incubated cells are selectively treated with cancer-specific drug compounds such as Lapatinib ditosylate, Gefitinib, Trastuzumab, Cetuximab, and Bevacizumab etc for cell responses study and drug screening. In this application, the structure of microsieve supporting rings can be utilized as physical walls of microwells. This feature can be utilized for multiple drug studies as in the microtiter plate.

### Example 5

For some applications, the fabricated SU-8 microsieve is integrated with a plastic holder as illustrated in FIG. 7. Three different designs are developed with either a simple outlet, an outlet with integrated Duckbill valve (DU 027.001 S, MiniValve, USA), or an outlet with embedded small holes. The duckbill is closed when the applied pressure is below its threshold pressure, whereas the embedded small holes will impose a surface tension force on the outlet. These structures are designed for fluid resistance control. When the applied pressure is lower than the design threshold force of the Duckbill valve or the embedded small holes, the fluid will be confined in the bottom chamber between the microsieve and outline. This feature would enable on-microsieve cell staining with reduced reagents. It can also be used for on-microsieve cell culture and drug response study.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A microsieve comprising two layers, wherein:
the first layer is a membrane layer having a plurality of micropores contained therein and a thickness of about 10 μm to about 100 μm; and
the second layer is a membrane support layer having a plurality of openings contained therein and a thickness of about 100 μm to about 500 μm, wherein the openings are larger in diameter than the micropores, and wherein at least one of the membrane layer or membrane support layer is formed of a SU-8 photoresist material.

2. The microsieve of claim 1,
wherein both the membrane layer and membrane support layer are formed of a SU-8 photoresist material.

3. The microsieve of claim 1,
wherein the openings are at least 10 times larger in diameter than the micropores.

4. The microsieve of claim 1,
wherein the membrane layer has a thickness of about 50 μm to about 100 μm.

5. The microsieve of claim 1,
wherein the membrane support layer has a thickness of about 200 μm to about 300 μm.

6. The microsieve of claim 1,
wherein each of the plurality of micropores has a pore diameter of about 5 μm to about 50 μm.

7. The microsieve of claim 6,
wherein each of the plurality of micropores has a pore diameter of about 10 μm, the membrane has a micropore density of about 5,000 micropores/mm².

8. The microsieve of claim 1,
wherein the microsieve has been surface-treated to decrease fluid resistance.

9. The microsieve of claim 1,
wherein the microsieve has been surface-coated with a metal layer.

10. A method of preparing a microsieve comprising two layers, wherein:
the first layer is a membrane layer having a plurality of micropores contained therein and a thickness of about 10 μm to about 100 μm; and
the second layer is a membrane support layer having a plurality of openings contained therein and a thickness of about 100 μm to about 500 μm, wherein the openings are larger in diameter than the micropores, and wherein at least one of the membrane layer or membrane support layer is formed of a SU-8 photoresist material, comprising:
providing a substrate;
coating the substrate with a first layer having a thickness of about 10 μm to about 100 μm;
patterning the first layer to form a plurality of micropores therein;
coating the patterned first layer with a second layer having a thickness of about 100 μm to about 500 μm; and
patterning the second layer to form a plurality of openings wherein the openings are larger in diameter than the micropores, and wherein at least one of the first layer or second layer is formed of a SU-8 photoresist material.

11. The method of claim 10,
wherein patterning the first layer comprises:
applying a photoresist mask that defines a pattern of dots corresponding to the micropores to be formed; and
exposing the first layer with the applied photoresist mask to UV light.

12. The method of claim 10,
wherein patterning the second layer comprises:
applying a photoresist mask that defines a pattern of shapes corresponding to the openings to be formed; and
exposing the second layer with the applied photoresist mask to UV light.

13. The method of claim 10,
further comprising developing the first layer and the second layer after patterning.

14. The method of claim 10,
further comprising coating the substrate with a lift-off resist layer prior to coating the first layer.

15. The method of claim 13,
further comprising removing lift-off resist layer and/or the substrate after developing.

16. A device for separating cells of a defined size from a fluid sample, where the device comprises:
an inlet module having an inlet for the fluid sample entry;
an outlet module having an outlet for the fluid sample exit;
a microsieve comprising two layers, wherein:
the first layer is a membrane layer having a plurality of micropores contained therein and a thickness of about 10 μm to about 100 μm; and
the second layer is a membrane support layer having a plurality of openings contained therein and a thickness of about 100 μm to about 500 μm, wherein the openings are larger in diameter than the micropores, and wherein at least one of the membrane layer or membrane support layer is formed of a SU-8 photoresist material, having micropores for retaining cells of a defined size arranged between the inlet module and the outlet module, wherein the inlet module, the outlet module, and the microsieve are fluidly connected to each other to allow the fluid sample to pass through from the inlet module to the outlet module.

17. A method of separating cells of a defined size from a fluid sample, comprising filtering the fluid sample suspected to comprise a cell to be separated through an inlet module of a device for separating cells of a defined size from a fluid sample, where the device comprises:
an inlet module having an inlet for the fluid sample entry;
an outlet module having an outlet for the fluid sample exit;
a microsieve comprising two layers, wherein:
the first layer is a membrane layer having a plurality of micropores contained therein and a thickness of about 10 μm to about 100 μm; and
the second layer is a membrane support layer having a plurality of openings contained therein and a thickness of about 100 μm to about 500 μm, wherein the openings are larger in diameter than the micropores, and wherein at least one of the membrane layer or membrane support layer is formed of a SU-8 photoresist material, having micropores for retaining cells of a defined size arranged between the inlet module and the outlet module, wherein the inlet module, the outlet module, and the microsieve are fluidly connected to each other to allow the fluid sample to pass through from the inlet module to the outlet module.

18. The method of claim 17,
wherein the fluid sample is selected from the group consisting of whole blood, urine, culture medium, and lysed tissue solution.

19. The method of claim 17,
wherein the cells to be detected are selected from the group consisting of circulating tumor cells, epithelial cells, cancer cells or cancer stem cells from lysed cancer tissue, cells comprised in a urine sample, and enrichment of cells from cell culture medium.

20. (canceled)

21. The method of claim 19, wherein the cells to be detected are circulating tumor cells separated from whole blood.
